(19) United States
(12) Patent Application Publication
(10) Pub. No.: US 2024/0320947 A1
(43) Pub. Date: Sep. 26, 2024

(54) INFORMATION PROCESSING DEVICE, PULSE WAVE CALCULATION METHOD, AND RECORDING MEDIUM

(71) Applicant: SHARP KABUSHIKI KAISHA, Sakai City (JP)

(72) Inventors: Ryota TOMIZAWA, Sakai City (JP); Yoshihisa ADACHI, Sakai City (JP); Tetsuya OKUMURA, Sakai City (JP)

(21) Appl. No.: 18/589,445

(22) Filed: Feb. 28, 2024

(30) Foreign Application Priority Data
Mar. 22, 2023 (JP) 2023-044926

(51) Int. Cl. G06V 10/25; G06T 7/62; G06V 10/74

(52) U.S. Cl. CPC G06V 10/25; G06T 7/62; G06V 10/761; G06V 2201/07

(57) ABSTRACT

An information processing device including: a region of interest specifying unit configured to specify a location of a region of interest regarding an image of each frame of a moving image captured of a living organism; a pixel value calculation unit configured to calculate, in an image of a first frame of the moving image, a plurality of first representative values of pixel values of pixels in locations of a plurality of regions of interest specified regarding images of a plurality of second frames including the image of the first frame; a pixel value combining unit configured to calculate a second representative value regarding the first frame by combining the plurality of first representative values; and a pulse wave calculation unit configured to calculate a pulse wave signal from a temporal change in the second representative value.

### FIELD OF THE INVENTION

The present disclosure relates to information processing devices, pulse wave calculation methods, and recording media. The present application claims the benefit of priority to Japanese Patent Application No. 2023-44926 filed in Japan on Mar. 22, 2023, the entire contents of which are incorporated herein by reference.

### BACKGROUND OF THE DISCLOSURE

PCT International Application Publication No. WO2019/203106 discloses a technique for, in calculating for a pulse wave from a video of a human face: detecting a face in each frame of the video; identifying a noise source that reduces pulse wave precision; specifying a region of interest and subregions into which the region of interest is divided; and producing a noise-restrained pulse wave signal by weighting the pulse wave signals calculated for each subregion using information on the noise source. In the technique disclosed in PCT International Application Publication No. WO2019/203106, when it is determined that there has been a rigid body motion or non-rigid body motion, the rigid body motion or non-rigid body motion is identified as the noise source, and the pulse wave is calculated without using the pixels in those portions of the region of interest where such a motion has been detected.

### SUMMARY

According to the technique disclosed in PCT International Application Publication No. WO2019/203106, the number of pixels used in the region of interest decreases when it is determined that there has been a rigid body motion or non-rigid body motion that is attributable to a displacement of a living organism that is the subject of image capturing over when the living organism has not moved. When the number of pixels in the region of interest used to calculate a pulse wave decreases, the S/N ratio falls, and therefore noise could increase across the data set. In addition, according to the technique disclosed in PCT International Application Publication No. WO2019/203106, the number of pixels changes in the region of interest in accordance with whether or not the living organism has moved, and therefore, the calculated pulse wave signal could include noise.

In view of these issues, the present disclosure, in an aspect thereof, has an object to provide an information processing device, a pulse wave calculation method, and a recording medium, any of which enables restraining a pulse wave signal from including noise due to a displacement of a living organism.

An information processing device in accordance with one embodiment of the present disclosure includes: a region of interest specifying unit configured to specify a location of a region of interest regarding an image of each frame of a moving image captured of a living organism; a pixel value calculation unit configured to calculate, in an image of a first frame of the moving image, a plurality of first representative values of pixel values of pixels in locations of a plurality of regions of interest specified regarding images of a plurality of second frames including the image of the first frame; a pixel value combining unit configured to calculate a second representative value regarding the first frame by combining the plurality of first representative values; and a pulse wave calculation unit configured to calculate a pulse wave signal from a temporal change in the second representative value.

A pulse wave calculation method in accordance with one embodiment of the present disclosure includes: a step of specifying a location of a region of interest regarding an image of each frame of a moving image captured of a living organism; a step of calculating, in an image of a first frame of the moving image, a plurality of first representative values of pixel values of pixels in locations of a plurality of regions of interest specified regarding images of a plurality of second frames including the image of the first frame; a step of calculating a second representative value regarding the first frame by combining the plurality of first representative values; and a step of calculating a pulse wave signal from a temporal change in the second representative value.

A computer-readable recording medium in accordance with one embodiment of the present disclosure contains a program configured to cause a computer to implement: a function of specifying a location of a region of interest regarding an image of each frame of a moving image captured of a living organism; a function of calculating, in an image of a first frame of the moving image, a plurality of first representative values of pixel values of pixels in locations of a plurality of regions of interest specified regarding images of a plurality of second frames including the image of the first frame; a function of calculating a second representative value regarding the first frame by combining the plurality of first representative values; and a function of calculating a pulse wave signal from a temporal change in the second representative value.

### BRIEF DESCRIPTION OF DRAWINGS

FIG. 1 is a diagram illustrating an exemplary use of an information processing device.

FIG. 2 is a block diagram of an exemplary structure of the information processing device in accordance with Embodiment 1.

FIG. 3 is a flow chart representing an exemplary operation of the information processing device in accordance with Embodiment 1.

FIG. 4A is a diagram illustrating exemplary locations of regions of interest in images of five second frames.

FIG. 4B is a diagram illustrating exemplary locations of five regions of interest shown as an example in FIG. 4A in the image of a first frame.

FIG. 5 is a diagram illustrating an exemplary time-series signal representing temporal changes in a second representative value.

FIG. 6 is a diagram illustrating an exemplary pulse wave signal calculated from the time-series signal shown as an example in FIG. 5.

FIG. 7 is a block diagram of an exemplary structure of an information processing device in accordance with Embodiment 2.

FIG. 8 is a flow chart representing an exemplary operation of the information processing device in accordance with Embodiment 2.

FIG. 9 is a flow chart representing an exemplary operation of the information processing device in accordance with Embodiment 2, which is a continuation of FIG. 8.

### DESCRIPTION OF EMBODIMENTS

### Embodiment 1

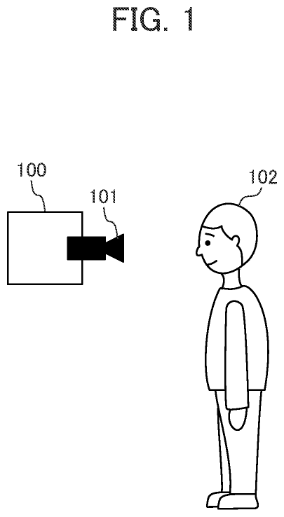

A description is given of Embodiment 1 with reference to FIGS. 1 to 6. Note that identical and equivalent elements in the drawings are denoted by the same reference numerals, and description thereof is not repeated.

FIG. 1 is a diagram illustrating an exemplary use of an information processing device 100. As shown as an example in FIG. 1, the information processing device 100 includes an imaging unit 101.

The information processing device 100 calculates a pulse wave signal representing a pulse wave from images acquired by the imaging unit 101. The information processing device 100 is, for example, a PC (personal computer), a smartphone, a tablet terminal, or a dedicated pulse wave measurement terminal. Throughout the present specification, a pulse wave is a time-series signal, representing changes in the volume of blood vessels, that is calculated from a time-series signal representing the pixel values of pixels contained in an image in relation to the same location on a body surface. Throughout the present specification, a pixel value is information representing the brightness of a pixel contained in an image and is, for example, a pixel value or a luminance value of a pixel for each of R (Red), G (Green), and B (Blue).

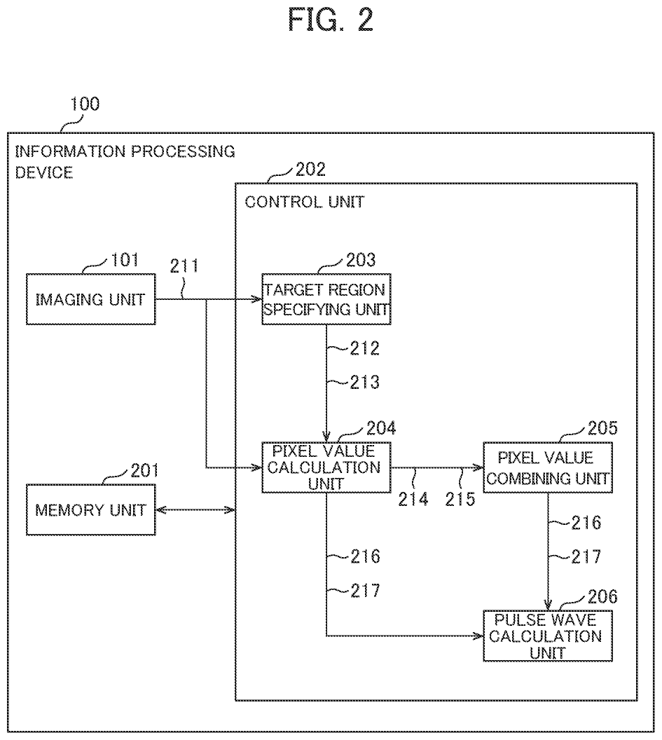

The imaging unit 101 captures images of a living organism 102 to acquire a moving image 211 (see FIG. 2). The moving image 211 contains images of the body surface of the living organism 102. For example, the imaging unit 101 is built around a CCD (charge coupled device) or a CMOS (complementary metal oxide semiconductor) image sensor. The imaging unit 101 may be built around a camera-use image sensor that includes RGB filters.

FIG. 2 is a block diagram of an exemplary structure of the information processing device 100 in accordance with the present embodiment. The information processing device 100 includes the imaging unit 101, a memory unit 201, and a control unit 202.

The imaging unit 101 captures images of the living organism 102 to acquire the moving image 211.

The memory unit 201 is a recording medium capable of recording, for example, various data and programs and built around, for example, a hard disk, an SSD (solid state drive), or a semiconductor memory device.

The control unit 202 performs various processes in accordance with the programs and data stored in the memory unit 201. The control unit 202 is realized by, for example, a CPU (central processing unit) or a like processor.

The control unit 202 includes a region of interest specifying unit 203, a pixel value calculation unit 204, a pixel value combining unit 205, and a pulse wave calculation unit 206.

The region of interest specifying unit 203 specifies the location of a region of interest regarding the image of each frame of the moving image 211. The region of interest is specified in an image of a body surface. For example, an image of a body surface is, for example, an image of a face, an image of a cheek, an image of a forehead, an image of a palm, an image of a wrist, or an image of a sole of a foot. Then, the region of interest specifying unit 203 outputs, to the pixel value calculation unit 204, a set of region of interest information 212 and identification information 213 regarding the image of each frame of the moving image 211. The region of interest information 212 represents the coordinates representing a region of interest regarding the image of each frame of the moving image 211. The identification information 213 is information for identifying the frame in which a region of interest is specified.

The pixel value calculation unit 204 calculates, in the image of a first frame, a plurality of first representative values 214 of the pixel values of pixels in the locations of a plurality of regions of interest specified regarding the images of a plurality of second frames including the image of the first frame of the moving image 211. When a plurality of second frames are selected regarding the first frame, the pixel value calculation unit 204 outputs a set of the plurality of first representative values 214 and a plurality of pieces of identification information 215 to the pixel value combining unit 205. The plurality of pieces of identification information 215 are information for identifying the first frame and the plurality of second frames.

In addition, when a plurality of second frames are not selected regarding the first frame, the pixel value calculation unit 204 calculates, as a second representative value 216, a representative value of the pixel values of pixels in the location of a region of interest specified regarding the image of the first frame. Then, when a plurality of second frames are not selected regarding the first frame, the pixel value calculation unit 204 outputs a set of the second representative value 216 and identification information 217 to the pulse wave calculation unit 206. The identification information 217 is information for identifying the first frame.

The pixel value combining unit 205 calculates the second representative value 216 regarding the first frame by combining the plurality of first representative values 214. The pixel value combining unit 205 outputs a set of the second representative value 216 and the identification information 217 to the pulse wave calculation unit 206.

The pulse wave calculation unit 206 calculates a pulse wave signal from temporal changes in the second representative value 216.

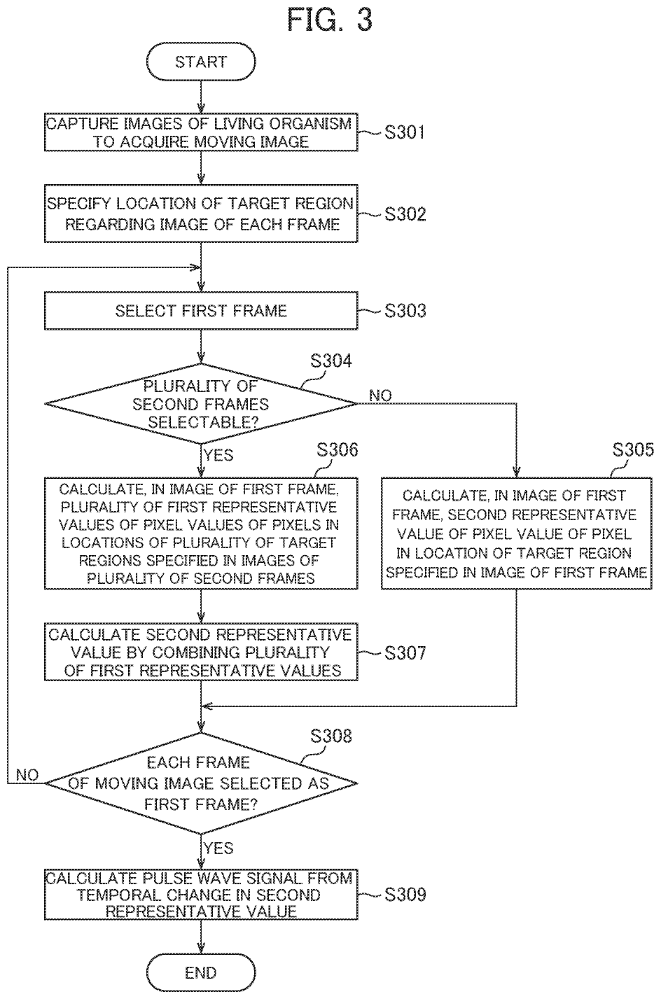

FIG. 3 is a flow chart representing an exemplary operation of the information processing device 100 in accordance with the present embodiment.

In step S301, the imaging unit 101 captures images of the living organism 102 to acquire the moving image 211. The imaging unit 101 outputs the image of each frame of the acquired moving image 211 to both the region of interest specifying unit 203 and the pixel value calculation unit 204. Note that the information processing device 100 may acquire the moving image 211 from a device other than the imaging unit 101. In other words, a device other than the imaging unit 101 may capture images of the living organism 102 to acquire the moving image 211. The device other than the imaging unit 101 may then transmit the acquired moving image 211 to the information processing device 100.

In step S302, the region of interest specifying unit 203 specifies the location of a region of interest regarding the image of each frame of the moving image 211. Then, the region of interest specifying unit 203 outputs, to the pixel value calculation unit 204, a set of the region of interest information 212 and the identification information 213 regarding the image of each frame of the moving image 211. For example, the identification information 213 represents the serial number of each frame of the moving image 211. Alternatively, the identification information 213 may represent a time elapsed since the start of the image capturing by the imaging unit 101, regarding a point in time at which each frame of the moving image 211 is acquired. As another alternative, the identification information 213 may represent a time at which each frame of the moving image 211 is acquired.

For instance, when the image of a frame of the moving image 211 contains an image of a face, the region of interest specifying unit 203 determines whether or not the image of the frame of the moving image 211 contains an image of a face, by detecting a feature such as an eye, a nose, and a mouth. As an example, suppose that the region of interest specifying unit 203 specifies a region of an image of a cheek as a region of interest. In such a case, when the image of a frame of the moving image 211 contains an image of a cheek, the region of interest specifying unit 203 specifies a region of interest in the location of an image of the detected cheek.

For instance, the region of interest specifying unit 203 specifies the location of the region of interest by detecting an image of a cheek, regarding the image of each frame of the moving image 211. Alternatively, when a region of interest has been specified regarding the image of a single frame of the moving image 211, the region of interest specifying unit 203 may track the region of interest by object tracking to specify the location of the region of interest regarding images of frames that follow this single frame.

For instance, the region of interest may have either a shape of a polygon surrounded by straight lines or a shape surrounded by curved lines. Alternatively, the region of interest may have a shape determined in accordance with the shape of a body part of the living organism. For example, when the region of interest specifying unit 203 specifies a region of interest using a polygon, the region of interest information 212 represents the coordinates of vertices of a polygon representing the region of interest. In addition, for example, when the region of interest specifying unit 203 specifies a region of interest using a circle, the region of interest information 212 represents the coordinates of the center of the circle representing the region of interest and the length of the radius representing the region of interest.

Alternatively, the region of interest information 212 may represent a matrix of logic values regarding the image of each frame of the moving image 211. The matrix of logic values represented by the region of interest information 212 indicates whether or not each pixel in the image is contained in the region of interest. In other words, the matrix of logic values represented by the region of interest information 212 is a matrix of the same size as the image of a frame of a moving image and indicates the location of the region of interest.

In step S303, the pixel value calculation unit 204 selects a first frame from a plurality of frames of the moving image 211 in time order.

In step S304, the pixel value calculation unit 204 determines whether or not a plurality of second frames including the first frame selected in step S303 can be selected from the plurality of frames of the moving image 211. Here, suppose that a plurality of second frames include the image of the first frame and represent a plurality of images captured in a prescribed time. In such a case, the pixel value calculation unit 204 determines whether or not it is possible to select the images of a plurality of frames that include the image of the first frame and that also have been captured in a prescribed time.

Alternatively, suppose that the images of the plurality of second frames include the image of the first frame and a plurality of successively captured images represent the images of the plurality of second frames. In such a case, the pixel value calculation unit 204 determines whether or not it is possible to select the images of a plurality of frames that include the image of the first frame and that also have been successively captured in a prescribed time.

In addition, as an example, suppose that the images of the plurality of second frames represent the image of the first frame and the image of at least one frame captured before the first frame. Then, suppose that the first frame represents the image of a frame that is captured first among the images of the plurality of frames of the moving image 211. In such a case, the pixel value calculation unit 204 cannot select a plurality of second frames from the plurality of frames of the moving image 211.

In addition, as an example, suppose that the images of the plurality of second frames represent the image of the first frame and the image of at least one frame captured after the first frame. Then, the first frame represents the image of a frame that is captured last among the images of the plurality of frames of the moving image 211. In such a case, the pixel value calculation unit 204 cannot select a plurality of second frames including the first frame from the plurality of frames of the moving image 211.

When a plurality of second frames cannot be selected in step S304, the pixel value calculation unit 204, in step S305, calculates, in the image of the first frame, the second representative value 216 of the pixel values of pixels in the location of a region of interest specified in the image of the first frame. For example, when the imaging unit 101 is built around a camera-use image sensor that includes RGB filters, the pixel value calculation unit 204, in step S305, calculates the second representative value 216 of a pixel value regarding R, G, and B pixels respectively in the image of the first frame. Then, the pixel value calculation unit 204 outputs a set of the second representative value 216 and the identification information 217 to the pulse wave calculation unit 206. The identification information 217 is information for identifying the first frame. The second representative value 216 is, for example, an average value, a median value, a maximum value, or a minimum value of the pixel value of each pixel in the region of interest.

On the other hand, when a plurality of second frames can be selected in step S304, the pixel value calculation unit 204, in step S306, calculates, in the image of the first frame, the plurality of first representative values 214 of the pixel values of pixels in the locations of a plurality of regions of interest specified in the images of the plurality of second frames.

For instance, when the imaging unit 101 is built around a camera-use image sensor that includes RGB filters, the pixel value calculation unit 204, in step S306, calculates, in the image of the first frame, the plurality of first representative values 214 regarding R, G, and B pixels respectively in the locations of a plurality of regions of interest specified in the images of the plurality of second frames. As an example, suppose that the number of the second frames is 10. In such a case, ten regions of interest that are specified in the images of ten frames that are the second frames are specified in the image of the first frame. Then, the pixel value calculation unit 204 calculates ten, first representative values 214 from the pixel values of pixels in the locations of the ten specified regions of interest, regarding R, G, and B pixels respectively. Then, the pixel value calculation unit 204 outputs a set of the plurality of first representative values 214 and the identification information 215 to the pixel value combining unit 205. The identification information 215 is information for identifying the first frame and the plurality of second frames.

In step S307, the pixel value combining unit 205 calculates the second representative value 216 regarding the first frame by combining the plurality of first representative values 214 calculated in the image of the same first frame. The second representative value 216 calculated in step S307 is, for example, an average value, a median value, a maximum value, or a minimum value of the plurality of first representative values 214.

For instance, when the imaging unit 101 is built around a camera-use image sensor that includes RGB filters, the pixel value combining unit 205, in step S307, calculates the second representative value 216 of pixel values regarding R, G, and B pixels respectively, in the image of the first frame. For example, when the number of the second frames is 10, the pixel value calculation unit 204 calculates ten, first representative values 214 regarding R, G, and B pixels respectively. In such a case, the pixel value combining unit 205 calculates the second representative value 216 by combining the ten calculated, first representative values 214 regarding R, G, and B pixels respectively. Alternatively, the pixel value combining unit 205 may calculate the second representative value 216 by weighting the plurality of first representative values 214 respectively and combining the plurality of weighted, first representative values 214.

In step S308, the pixel value calculation unit 204 determines whether or not each frame of the moving image 211 has been selected as a first frame. When each frame of the moving image 211 has not been selected as a first frame in step S308, the control unit 202 returns the process to step S303. On the other hand, when each frame of the moving image 211 has been selected as a first frame in step S308, the control unit 202 forwards the process to step S309.

In step S309, the pulse wave calculation unit 206 calculates a pulse wave signal from temporal changes in the second representative value 216. Specifically, the pulse wave calculation unit 206 calculates a pulse wave signal from temporal changes in the second representative value 216. For example, the pulse wave calculation unit 206 processes a signal representing temporal changes in the second representative value 216 by multivariable analysis such as principal component analysis or independent component analysis and then calculates the result of this process as a pulse wave signal.

For instance, when the pixel value combining unit 205 has combined the plurality of first representative values 214 regarding R, G, and B pixels respectively to calculate the second representative value 216, the pulse wave calculation unit 206 calculates a time-series signal for the second representative value 216 regarding R, G, and B pixels respectively. In other words, when the pixel value combining unit 205 has calculated the second representative value 216 regarding R, G, and B pixels respectively, the pulse wave calculation unit 206 calculates three time-series signals representing temporal changes in the second representative value 216. Then, the pulse wave calculation unit 206 calculates a pulse wave signal from the three calculated time-series signals.

For instance, suppose that the pixel value calculation unit 204 selects an image Ft of the first frame and the image of at least one frame captured before the first frame as the images of the plurality of second frames. In such a case, in step S309, the pulse wave calculation unit 206 uses the second representative value 216 calculated in step S304 regarding the image of the frame captured first among the images of the plurality of frames of the moving image 211 and uses the second representative value 216 calculated in step S307 regarding the images of the second and subsequent frames.

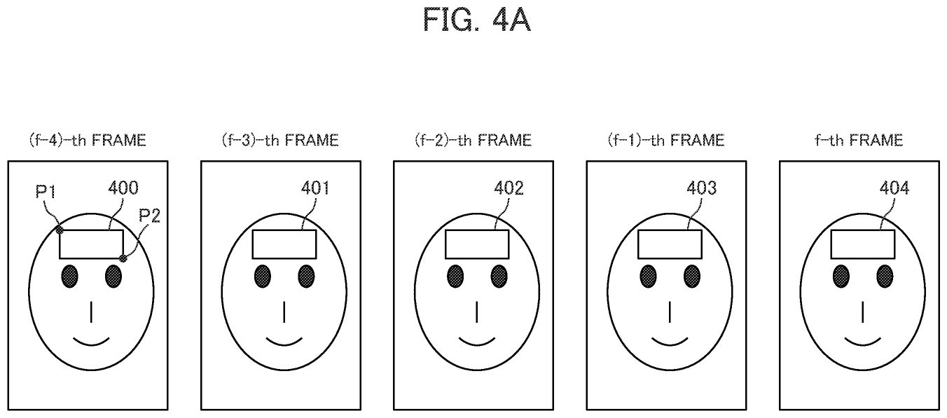

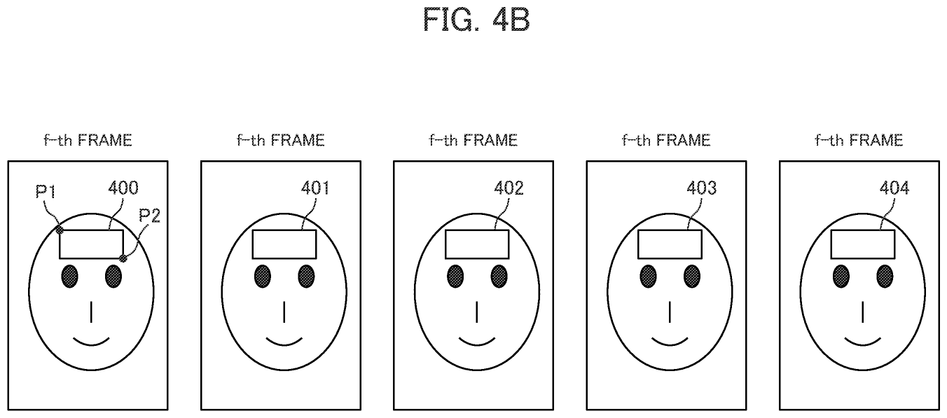

FIG. 4A illustrates exemplary locations of regions of interest 400 to 404 in images of five second frames, which are from the (f−4)-th frames to the f-th frame. FIG. 4B illustrates exemplary locations of the regions of interest 400 to 404 regions shown as an example in FIG. 4A in the image of the first frame, which is the f-th frame.

For instance, suppose that the region of interest 400 specified in the image of the (f−4)-th frame is rectangular. Then, suppose that in the region of interest 400, the upper left vertex has coordinates P1, (x1, y1), and the lower right vertex has coordinates P2, (x2, y2). In such a case, in the image of the f-th frame, the pixel value calculation unit 204 calculates the first representative value 214 of the pixel values of pixels in the locations of the rectangular region of interest in which the upper left vertex has coordinates P1, (x1, y1), and the lower right vertex has coordinates P2, (x2, y2). Similarly, the pixel value calculation unit 204 calculates four first representative values 214 of the pixel values of pixels in the locations of the region of interest 401 through the region of interest 404 specified in the (f−3)-th frame through the f-th frame. Then, the pixel value combining unit 205 calculates the second representative value 216 of the five first representative values 214 of the pixel values of pixels in locations of the region of interest 400 through the region of interest 404.

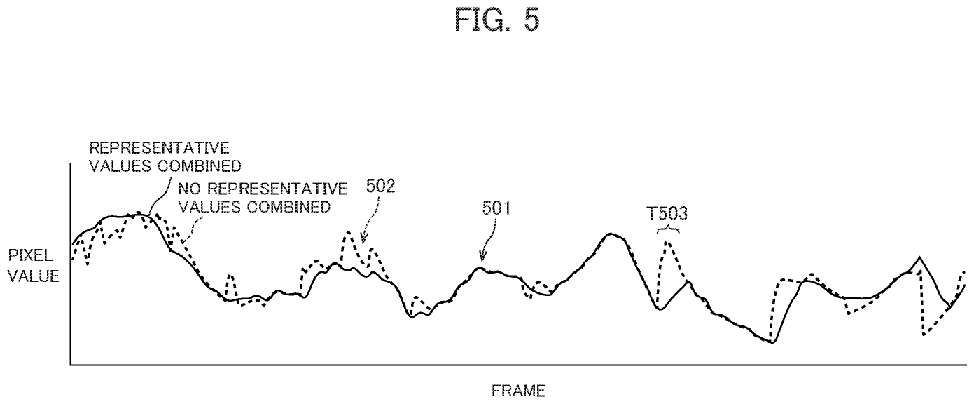

FIG. 5 is a diagram illustrating an exemplary time-series signal 501 representing temporal changes in the second representative value 216. Furthermore, FIG. 5 illustrates an exemplary time-series signal 502 representing temporal changes in a representative value of the pixel values of pixels in the location of a region of interest specified in the image of each frame of a moving image as a comparative example.

For instance, suppose that the location of a region of interest has moved one pixel from the location of the region of interest specified in the preceding frame due to a small displacement of the living organism 102. In such a case, the one-pixel displacement of the region of interest can abruptly change the distribution of the pixel values of pixels contained in the region of interest. When the distribution of the pixel values of pixels contained in the region of interest has abruptly changed, the representative value of the pixel values of pixels in a location of the region of interest abruptly changes as shown as an example in period T503 on the time-series signal 502.

Meanwhile, the second representative value 216 changes more gently on the time-series signal 501 than on the time-series signal 502 in period T503. In other words, the information processing device 100 is capable of restraining abrupt changes in the representative value of pixel values.

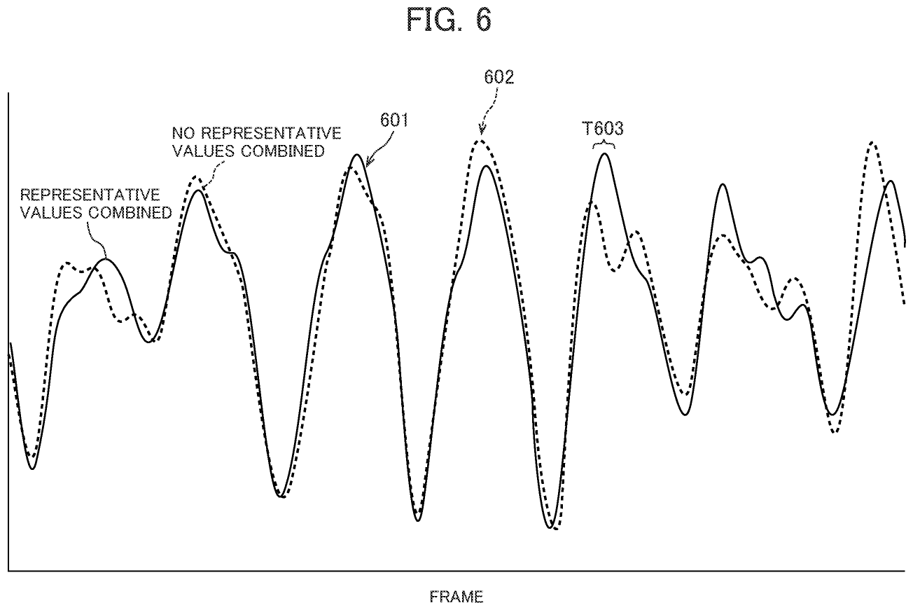

FIG. 6 is a diagram illustrating an exemplary pulse wave signal 601 calculated from the time-series signal 501 shown as an example in FIG. 5. FIG. 6 also illustrates an exemplary pulse wave signal 602 calculated from the time-series signal 502 shown as a comparative example in FIG. 5. Period T603 shown as an example in FIG. 6 is the same period as period T503 shown as an example in FIG. 5.

An abrupt change in the representative value of the pixel values of pixels in the location of a region of interest could result in producing noise on the pulse wave signal calculated from temporal changes in the representative value. For example, when the representative value of the pixel values of pixels in the location of a region of interest has changed abruptly as shown as an example on the time-series signal 502 in period T503, the pulse wave signal 602 in period T603 changes more abruptly than the pulse wave signal 601 in period T603. Meanwhile, the information processing device 100 calculates a pulse wave signal from temporal changes in the second representative value 216 calculated by combining the plurality of first representative values 214. Hence, the information processing device 100 can restrain variations in the pixel values of pixels in the region of interest caused by a slight displacement of the living organism 102 from resulting in producing noise on the calculated pulse wave signal.

### Embodiment 2

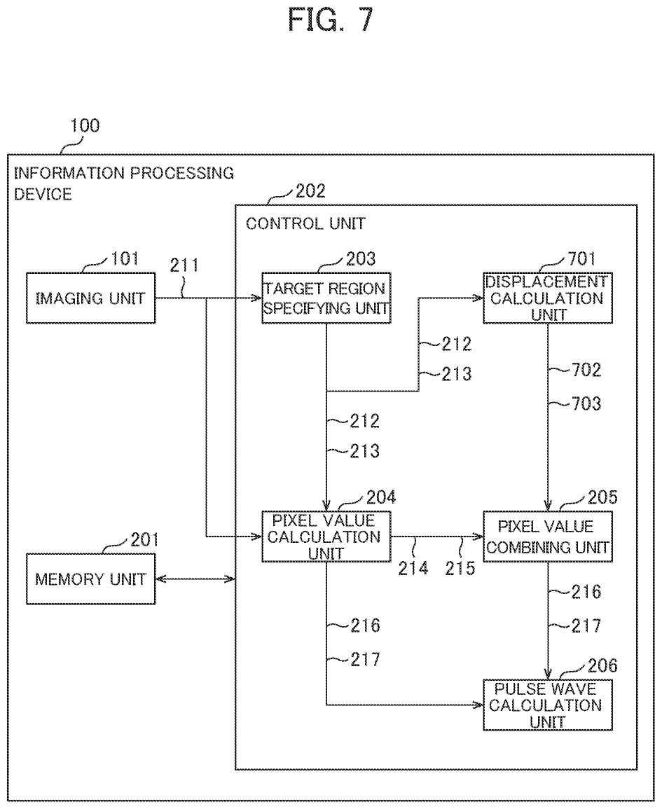

A description is given of Embodiment 2 with reference to FIGS. 7 to 9. Note that identical and equivalent elements in the drawings are denoted by the same reference numerals, and description thereof is not repeated. The members and processes of the present embodiment that have practically the same arrangement and function as the members and processes of another embodiment are indicated by the same reference numerals, and description thereof is omitted. The description will focus on differences from the other embodiment.

FIG. 7 is a block diagram of an exemplary structure of the information processing device 100 in accordance with the present embodiment. The information processing device 100 shown as an example in FIG. 7 differs from the information processing device 100 shown as an example in FIG. 2 in that the information processing device 100 shown as an example in FIG. 7 includes a displacement calculation unit 701.

The displacement calculation unit 701 calculates a displacement 702 between a first region of interest specified regarding the image of the first frame and a second region of interest specified regarding the image of one of the plurality of second frames other than the first frame. The displacement calculation unit 701 outputs, to the pixel value combining unit 205, a set of a displacement 702 and identification information 703 regarding each one of the plurality of second frames other than the first frame. The identification information 703 is information for identifying a frame in which the second region of interest that is used to calculate the displacement 702 is specified. The displacement 702 is calculated on the basis of at least any one of factors selected from the group consisting of a difference in location between the first region of interest and the second region of interest, a difference in area between the first region of interest and the second region of interest, and a rotation angle between the first region of interest and the second region of interest.

The pixel value combining unit 205 in accordance with the present embodiment gives a weight to each of the plurality of first representative values 214 in accordance with the displacement 702. Note that the images of the plurality of second frames may represent a plurality of images captured in a time determined in accordance with the displacement 702.

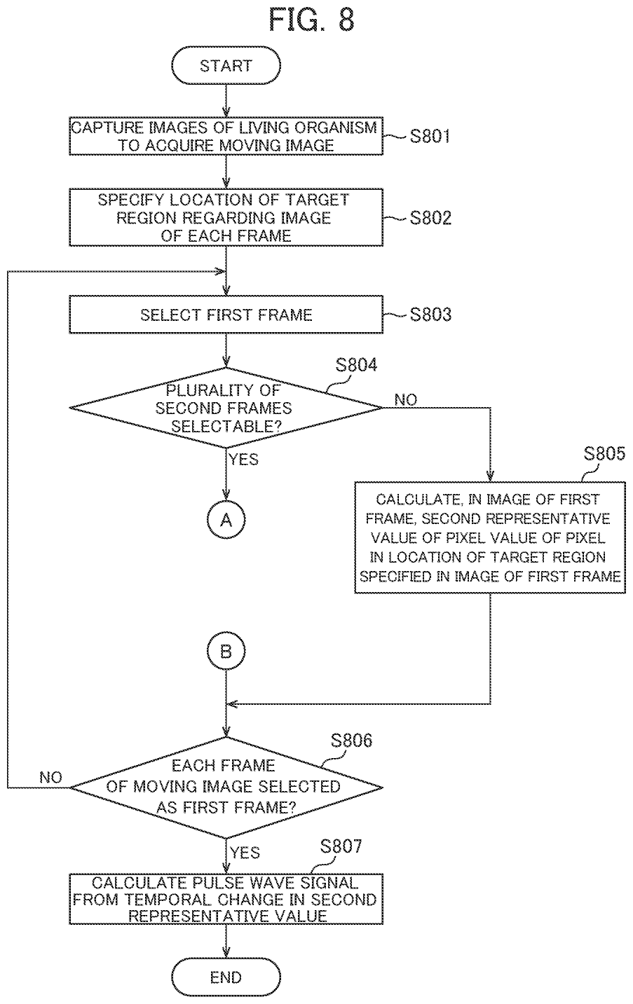

FIG. 8 is a flow chart representing an exemplary operation of the information processing device 100 in accordance with the present embodiment. Step S801 to step S804 are identical to step S301 to step S304 shown as an example in FIG. 3, and detailed description thereof is therefore omitted.

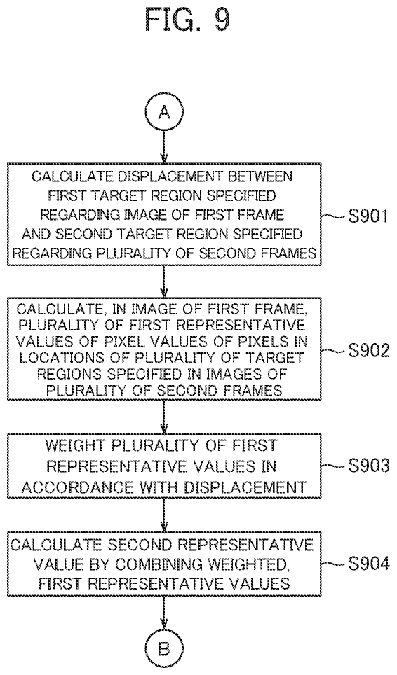

When the plurality of second frames cannot be selected in step S804, the control unit 202 forwards the process to step S805. Step S805 is identical to step S305 shown as an example in FIG. 3, and detailed description thereof is therefore omitted. Then, the control unit 202 forwards the process to step S806. Step S806 to step S807 are identified to step S308 to step S309 shown as an example in FIG. 3, and detailed description thereof is therefore omitted. On the other hand, when the plurality of second frames can be selected in step S804, the control unit 202 forwards the process to step S901 shown as an example in FIG. 9.

A description is given next of an operation of the information processing device 100 in accordance with the present embodiment with reference to FIG. 9.

In step S901, the displacement calculation unit 701 calculates the displacement 702 between the first region of interest specified regarding the image of the first frame that is selected in step S803 shown as an example in FIG. 8 and the second region of interest specified regarding the image of one of the plurality of second frames, other than the first frame, that is selected in step S804 shown as an example in FIG. 8. In other words, the displacement calculation unit 701 calculates the displacement 702 between the first region of interest and the second region of interest regarding each one of the plurality of second frames other than the first frame. Then, the displacement calculation unit 701 outputs, to the pixel value combining unit 205, a set of the displacement 702 and the identification information 703 regarding each one of the plurality of second frames other than the first frame. The identification information 703 is information for identifying a frame for which the displacement 702 is calculated.

The displacement 702 is calculated on the basis of at least any one of factors selected from the group consisting of a difference in location between the first region of interest and the second region of interest, a difference in area between the first region of interest and the second region of interest, and a rotation angle between the first region of interest and the second region of interest.

For instance, the “difference in location between the first region of interest and the second region of interest” refers to the distance between the coordinates of the center of the first region of interest and the coordinates of the center of the second region of interest. For example, when the first region of interest is polygonal, the “center of the first region of interest” refers to the center of gravity of the first region of interest. Likewise, when the second region of interest is polygonal, the “center of the second region of interest” refers to the center of gravity of the second region of interest.

In addition, for example, the “difference in area between the first region of interest and the second region of interest” refers to the differential value between the area of the first region of interest and the area of the second region of interest. Alternatively, the “difference in area between the first region of interest and the second region of interest” may represent the ratio of the area of the second region of interest to the area of the first region of interest.

In addition, for example, the “rotation angle between the first region of interest and the second region of interest” refers to the rotation angle calculated from, for example, an optical flow of corresponding pixels between the pixels contained in the first region of interest and the pixels contained in the second region of interest. Note that the displacement calculation unit 701 needs only to be capable of calculating the rotation angle between the first region of interest and the second region of interest, and the calculation method is not particularly limited.

In step S902, the pixel value calculation unit 204 calculates, in the image of the first frame, the plurality of first representative values 214 for the pixel values of pixels in the locations of a plurality of regions of interest specified in the images of the plurality of second frames. Step S902 is identical to step S306 shown as an example in FIG. 3, and detailed description thereof is therefore omitted.

In step S903, the pixel value combining unit 205 gives a weight to each of the plurality of first representative values 214 in accordance with the displacement 702. Suppose, as an example, that the living organism 102 moved between a point in time when an image of the first frame in which the first region of interest was specified was captured and a point in time when an image of the frame in which the second region of interest was specified was captured. In such a case, suppose that the displacement 702 between the location of the first region of interest and the location of the second region of interest is relatively large in the image of the first frame. Accordingly, for example, the pixel value combining unit 205 gives a relatively light weight to the first representative values 214 in locations of the second region of interest for which the displacement 702 is relatively large.

In step S904, the pixel value combining unit 205 calculates the second representative value 216 by combining the plurality of first representative values 214 weighted in step S903. Then, the control unit 202 forwards the process to step S806 shown as an example in FIG. 8.

Suppose, as an example, that the pixel value combining unit 205 gives a relatively light weight to the first representative values 214 in locations of the second region of interest for which the displacement 702 is relatively large. Hence, the pixel value combining unit 205 can calculate the second representative value 216 without having to give importance to the first representative values 214 calculated in those locations of the plurality of regions of interest specified in the plurality of second frames which are relative distanced from the location of the first region of interest.

A displacement of the living organism 102 could increase variations in the locations of the regions of interest specified in the second frames, which may increase noise on the pulse wave signal. However, the information processing device 100 in accordance with the present embodiment calculates the second representative value 216 by combining the plurality of first representative values 214 weighted in accordance with the displacement of the region of interest. Therefore, the information processing device 100 in accordance with the present embodiment is capable of restraining utilizing the pixel values of pixels that are irrelevant to the calculation of a pulse wave signal due to a displacement of the living organism 102, as well as achieving the same advantageous effects as the information processing device 100 in accordance with Embodiment 1.

The processes performed in the foregoing embodiments are not limited to the processes described as an example in the embodiments. The functional blocks described above may be implemented by logic circuits (hardware) fabricated, for example, in the form of integrated circuits or by software run by a CPU. The embodiments may be implemented by a plurality of computers. For example, the processes performed by the functional blocks of the control unit 202 in the information processing device 100 may be partially implemented by another computer or entirely implemented by a plurality of computers.

The present disclosure is not limited to the description of the embodiments and examples above. Any structure detailed in the embodiments and examples may be replaced by a practically identical structure, a structure that delivers practically the same effect and function, or a structure that delivers practically the same purpose. Embodiments based on a proper combination of technical means disclosed in different embodiments are encompassed in the technical scope of the present disclosure. Furthermore, new technological features can be created by combining different technical means disclosed in the embodiments.

What is claimed is:

1. An information processing device comprising:
a region of interest specifying unit configured to specify a location of a region of interest regarding an image of each frame of a moving image captured of a living organism;
a pixel value calculation unit configured to calculate, in an image of a first frame of the moving image, a plurality of first representative values of pixel values of pixels in locations of a plurality of regions of interest specified regarding images of a plurality of second frames including the image of the first frame;
a pixel value combining unit configured to calculate a second representative value regarding the first frame by combining the plurality of first representative values; and
a pulse wave calculation unit configured to calculate a pulse wave signal from a temporal change in the second representative value.

2. The information processing device according to claim 1, further comprising an imaging unit configured to acquire the moving image by capturing images of the living organism.

3. The information processing device according to claim 1, wherein the second representative value is calculated by the pixel value combining unit when the plurality of second frames are selected regarding the first frame and is a representative value of pixel values of pixels in a location of a region of interest specified regarding the image of the first frame when the plurality of second frames are not selected regarding the first frame.

4. The information processing device according to claim 1, wherein the images of the plurality of second frames represent a plurality of images captured in a prescribed time.

5. The information processing device according to claim 1, wherein the images of the plurality of second frames represent a plurality of successively captured images.

6. The information processing device according to claim 1, wherein the images of the plurality of second frames represent the image of the first frame and an image of at least one frame captured before the first frame.

7. The information processing device according to claim 1, wherein the images of the plurality of second frames represent the image of the first frame and an image of at least one frame captured after the first frame.

8. The information processing device according to claim 1, wherein the pixel value combining unit calculates the second representative value by weighting the plurality of first representative values and combining the plurality of weighted, first representative values.

9. The information processing device according to claim 8, further comprising a displacement calculation unit configured to calculate a displacement between a first region of interest specified regarding the image of the first frame and a second region of interest specified regarding an image of one of the plurality of second frames other than the first frame, wherein the pixel value combining unit weights the plurality of first representative values in accordance with the displacement.

10. The information processing device according to claim 9, wherein the displacement is calculated based on at least any selected from the group consisting of a difference in location between the first region of interest and the second region of interest, a difference in area between the first region of interest and the second region of interest, and a rotation angle between the first region of interest and the second region of interest.

11. The information processing device according to claim 9, wherein the images of the plurality of second frames represent a plurality of images captured in a time determined in accordance with the displacement.

12. A pulse wave calculation method comprising:
a step of specifying a location of a region of interest regarding an image of each frame of a moving image captured of a living organism;
a step of calculating, in an image of a first frame of the moving image, a plurality of first representative values of pixel values of pixels in locations of a plurality of regions of interest specified regarding images of a plurality of second frames including the image of the first frame;
a step of calculating a second representative value regarding the first frame by combining the plurality of first representative values; and
a step of calculating a pulse wave signal from a temporal change in the second representative value.

13. A computer-readable recording medium containing a program configured to cause a computer to implement:
a function of specifying a location of a region of interest regarding an image of each frame of a moving image captured of a living organism;
a function of calculating, in an image of a first frame of the moving image, a plurality of first representative values of pixel values of pixels in locations of a plurality of regions of interest specified regarding images of a plurality of second frames including the image of the first frame;
a function of calculating a second representative value regarding the first frame by combining the plurality of first representative values; and
a function of calculating a pulse wave signal from a temporal change in the second representative value.

14. The information processing device according to claim 2, wherein the second representative value is calculated by the pixel value combining unit when the plurality of second frames are selected regarding the first frame and is a representative value of pixel values of pixels in a location of a region of interest specified regarding the image of the first frame when the plurality of second frames are not selected regarding the first frame.

15. The information processing device according to claim 2, wherein the images of the plurality of second frames represent a plurality of images captured in a prescribed time.

16. The information processing device according to claim 2, wherein the images of the plurality of second frames represent a plurality of successively captured images.

17. The information processing device according to claim 2, wherein the images of the plurality of second frames represent the image of the first frame and an image of at least one frame captured before the first frame.

18. The information processing device according to claim 2, wherein the images of the plurality of second frames represent the image of the first frame and an image of at least one frame captured after the first frame.

19. The information processing device according to claim 2, wherein the pixel value combining unit calculates the second representative value by weighting the plurality of first representative values and combining the plurality of weighted, first representative values.

20. The information processing device according to claim 19, further comprising a displacement calculation unit configured to calculate a displacement between a first region of interest specified regarding the image of the first frame and a second region of interest specified regarding an image of one of the plurality of second frames other than the first frame, wherein the pixel value combining unit weights the plurality of first representative values in accordance with the displacement.
